(19) United States
(12) Patent Application Publication
(10) Pub. No.: US 2020/0046891 A1
(43) Pub. Date: Feb. 13, 2020

(54) FILTER CONTAINER FOR SEPARATING CELLS AND FILTER DEVICE FOR SEPARATING CELLS

(71) Applicant: KANEKA CORPORATION, Osaka (JP)

(72) Inventors: Keita Yamashita, Osaka (JP); Masahiro Kojima, Osaka (JP); Koshin Ushizaki, Osaka (JP)

(73) Assignee: KANEKA CORPORATION, Osaka (JP)

(21) Appl. No.: 16/655,949

(22) Filed: Oct. 17, 2019

Related U.S. Application Data
(63) Continuation of application No. PCT/JP2018/015879, filed on Apr. 17, 2018.

(30) Foreign Application Priority Data
Apr. 17, 2017 (JP) 2017-081617

(51) Int. Cl. A61M 1/36; A61M 1/02; A61M 1/34; A61M 1/16; A61M 1/30; C12N 5/0784; C12N 5/0786

(52) U.S. Cl. CPC A61M 1/3633; A61M 1/0259; A61M 1/34; A61M 1/3679; A61M 1/029; C12N 5/0645; A61M 1/0281; A61M 1/1672; A61M 1/304; C12N 5/0639; A61M 1/0218

(57) ABSTRACT

A filter container for separating cells includes a tubular filter member-housing part having an opening at each longitudinal end, a liquid inlet port on a first opening end side of the filter member-housing part, a liquid outlet port on a second opening end side of the filter member-housing part, a first filter-pressing part on the first opening end side, and a second filter-pressing part on the second opening end side. The first filter-pressing part includes three or more first protrusions, and the ratio of an area of a polygon formed by individually connecting the tips of the three or more first protrusions to a cross-sectional area of an inner space of the filter member-housing part at the first protrusion position is controlled within a preset range.

### TECHNICAL FIELD

One or more embodiments of the present invention relate to a filter container for separating cells, a filter device for separating cells in which a filter member is housed in the filter container for separating cells, a method for obtaining a cell-containing liquid including a step in which the filter device for separating cells is used, and a method for producing a dendritic cell-containing liquid including a step in which the filter device for separating cells is used.

### BACKGROUND

Recently, rapid developments in hematology and scientific technology have contributed to the wide spread of treatment styles that involve separating only a blood fraction necessary for the treatment from a biological fluid such as whole blood, bone marrow, umbilical cord blood, or a tissue extract, and then administering it to a patient to have improved therapeutic effects, and that do not involve administering fractions unnecessary for the treatment, thereby inhibiting side effects.

For example, blood transfusion is one of those treatment styles. Red blood cell products are blood products used in case of hemorrhage, the lack of red blood cells, or the lack of oxygen caused by reduced function of red blood cells. For red blood cell products, white blood cells, which may induce side effect such as an abnormal immunoreaction or graft versus host disease (GVHD), are unnecessary. Thus, white blood cells should be removed from red blood cell products by using a filter. In some cases, not only white blood cells but also platelets are removed.

On the other hand, platelet products are blood products used to patients with hemorrhage or hemorrhagic tendencies due to the lack of a blood coagulation factor. In order to prepare platelet products, unnecessary cells or components other than platelets are removed by centrifugal separation, and only desired platelet components are collected.

Also, hematopoietic stem cell transplants have recently become popular as treatment for leukemia. In the hematopoietic stem cell transplants, employed is a method in which a white blood cell group including hematopoietic stem cells required for the treatment is separated and administered. As a source of the hematopoietic stem cells, umbilical cord blood has attracted attention in addition to bone marrow and peripheral blood because of its advantages such as small burden on donors and high proliferative ability, or the like. Additionally, it has been recently suggested that menstrual blood is also rich in stem cells. Due to this reason, there is also a possibility of using menstrual blood, which has hitherto gone to waste, as a valuable source of stem cells. Furthermore, as a treatment of solid cancer, dendritic cell therapy is widely carried out. For dendritic cell therapy, there is a case in which a cell group containing monocytes, which are the basis of a treatment, is separated from blood or cell culture liquid of a patient.

With regard to the source of transplantation treatment like bone marrow and peripheral blood, it is desired that white blood cells are administered after they are separated and purified by removing unnecessary cells. Furthermore, based on the necessity for cryopreservation until use, white blood cells are separated and purified for the purpose of preventing red blood cell hemolysis that may be caused by cryopreservation.

As a method for separating cells, a method of collecting white blood cells by using a filter material capturing only the white blood cells without capturing red blood cells and platelets is also recently reported (see, Patent Document 1, Patent Document 2, and Patent Document 3). Conventionally, the separational operation has been performed by centrifugal separation or density gradient centrifuge using a specific gravity liquid, but use of a filter has an advantage that the operation can be simplified or large-size cell processing facilities are not required. However, when cell separation is actually carried out, there is a case in which, with unknown reasons, blood does not easily pass through inside the cell separating material (filter member) to be filled in a filter material, or cells of desired type cannot be collected at a sufficiently high collection rate.
Patent Document 1: Japanese Unexamined Patent Application (Translation of PCT Application), Publication No. 2001-518792
Patent Document 2: PCT International Publication No. WO98/32840
Patent Document 3: Japanese Unexamined Patent Application, Publication No. H10-313855

### SUMMARY

Under the circumstances described in the above, one or more embodiments of the present invention provide a filter container for separating cells, the filter container holding a filter member housed therein and thus providing a filter device for separating cells by which cells can be collected at a high collection rate, a filter device for separating cells in which a filter member is housed in the filter container for separating cells, a method for obtaining a cell-containing liquid including a step in which the filter device for separating cells is used, and a method for producing a dendritic cell-containing liquid including a step in which the filter device for separating cells is used.

Inventors of the present invention conducted intensive studies. As a result, it was found that, for a filter container for separating cells which has a liquid inlet port, a liquid outlet port, a tubular filter member-housing part, a first filter-pressing part, and a second filter-pressing part, if the first filter-pressing part is configured of three or more first protrusions and the ratio of the area of a polygon formed by connecting each tip of the three or more first protrusions to the cross-sectional area of the inner space of the filter member-housing part at the first protrusion position is controlled within a preset range, and one or more embodiments of the present invention are completed accordingly.

Namely, the summary of one or more embodiments of the present invention is as described below.

[1] A filter container for separating cells, including a liquid inlet port, a liquid outlet port, a filter member-housing part, a first filter-pressing part, and a second filter-pressing part, in which

the filter member-housing part is a tubular member having an opening at both ends,
the liquid inlet port is provided on one opening end side (“first opening end side”) of the filter member-housing part,
the liquid outlet port is provided on another opening end side (“second opening end side”) of the filter member-housing part,
the first filter-pressing part is configured, at a position on the liquid inlet port side of the filter member-housing part, of three or more first protrusions which protrude from an inner wall face of the filter member-housing part or from the vicinity of the inner wall face toward a center or approximate center of a cross-section of an inner space of the filter member-housing part,
the first filter-pressing part is disposed such that, when the filter member is housed in the filter member-housing part, the first filter-pressing part is in contact with the filter member while deforming the filter member,
the second filter-pressing part is disposed at a position on the liquid outlet port side of the filter member-housing part such that the filter member is inserted by the first filter-pressing part and the second filter-pressing part, and
when the area of a polygon formed by connecting each tip of the three or more first protrusions constituting the first filter-pressing part is A1 and the cross-sectional area of an inner space of the filter member-housing part at the first protrusion position is A2, the area ratio R1 that is calculated by the following equation is 6 to 50%:

Area ratio R1(%)=A1/A2×100.

[2] The filter container for separating cells described in [1], in which the second filter-pressing part is configured, at the position on the liquid outlet port side of the filter member-housing part, of three or more second protrusions which protrude from an inner wall face of the filter member-housing part or from the vicinity of the inner wall face toward a center or approximate center of a cross-section of an inner space of the filter member-housing part.

[3] The filter container for separating cells described in [2], in which, when the area of a polygon formed by connecting the tips of the three or more second protrusions constituting the second filter-pressing part is A3 and the cross-sectional area of an inner space of the filter member-housing part at the second protrusion position is A4, the area ratio R2 that is calculated by the following equation is 6 to 50%:

Area ratio R2(%)=A3/A4×100.

[4] The filter container for separating cells described in any one of [1] to [3], in which the area ratio R1 is 6 to 40%.

[5] The filter container for separating cells described in [3], in which the area ratio R2 is 6 to 40%.

[6] The filter container for separating cells described in any one of [1] to [5], in which the number of the first protrusions is 5 or more and 12 or less.

[7] The filter container for separating cells described in [3] or [5], in which the number of the second protrusions is 5 or more and 12 or less.

[8] A filter device for separating cells, including the filter container for separating cells described in any one of [1] to [7] and a filter member in which the filter member is housed in a filter member-housing part.

[9] The filter device for separating cells described in [8], in which the filter member consists of a non-woven fabric.

[10] A method for obtaining a cell-containing liquid containing monocytes, including:

capturing the monocytes by a filter member by supplying, from a liquid inlet port, a crude cell-containing liquid containing monocytes and small cells, which are cells having smaller average size than monocytes, to an inside of the filter device for separating cells described in [8] or [9] followed by passing the crude cell-containing liquid through the filter device for separating cells;
yielding the cell-containing liquid by releasing, in a collection liquid, the monocytes captured by the filter member by supplying a collection liquid to the inside of the filter device for separating cells; and
collecting the cell-containing liquid from the inside of the filter device for separating cells.

[11] The method described in [10], in which the collection liquid is supplied from a liquid outlet port and the cell-containing liquid is collected from a liquid inlet port.

[12] A method for producing a dendritic cell-containing liquid used for dendritic cell therapy, including:

capturing monocytes by a filter member by supplying, from a liquid inlet port, a crude cell-containing liquid containing monocytes and small cells, which are cells having smaller average size than monocytes, to an inside of the filter device for separating cells described in [8] or [9] followed by passing the crude cell-containing liquid through the filter device for separating cells;
yielding a cell-containing liquid by releasing, in a collection liquid, the monocytes captured by the filter member by supplying a collection liquid to the inside of the filter device for separating cells;
collecting the cell-containing liquid from the inside of the filter device for separating cells; and
differentiating the monocytes contained in the cell-containing liquid into dendritic cells.

[13] The method described in [12], in which the collection liquid is supplied from a liquid outlet port and the cell-containing liquid is collected from a liquid inlet port.

According to one or more embodiments of the present invention, a filter container for separating cells, the filter container holding a filter member housed therein and thus providing a filter device for separating cells by which cells can be collected at a high collection rate, a filter device for separating cells in which a filter member is housed in the filter container for separating cells, a method for obtaining a cell-containing liquid including a step in which the filter device for separating cells is used, and a method for producing a dendritic cell-containing liquid including a step in which the filter device for separating cells is used can be provided.

### BRIEF DESCRIPTION OF THE DRAWINGS

FIG. 1 is a diagram illustrating an outline of an exemplary filter container for separating cells according to one or more embodiments.

FIGS. 2(a) and 2(b) are a diagram illustrating cross-sections of an exemplary filter device for separating cells in a state in which a filter member is housed according to one or more embodiments.

FIG. 3 is a diagram illustrating an outline of an exemplary nozzle-attached inner lid according to one or more embodiments.

FIGS. 4(a)-4(d) are diagrams schematically illustrating variations of protrusion mode of a first protrusion in a first filter-pressing part according to one or more embodiments.

FIG. 5 is a diagram illustrating an outline of a filter device for separating cells according to one or more embodiments, which is used in Examples and Comparative Examples, in exploded state.

FIG. 6 is a diagram illustrating an outline of a circuit of a device for separating cells according to one or more embodiments, which is used in Examples and Comparative Examples.

### DETAILED DESCRIPTION OF THE EMBODIMENTS

Hereinbelow, one or more embodiments of the present invention are described in view of the drawings.

### <<Filter Container for Separating Cells>>

A filter container 2 for separating cells has a liquid inlet port 9, a liquid outlet port 10, a filter member-housing part 3, a first filter-pressing part 12, and a second filter-pressing part 13. Hereinbelow, the filter container for separating cells is also simply described as “container”. As a filter member 11 composed of a non-woven fabric or the like is housed in the filter container 2 for separating cells, a filter device 1 for separating cells is constituted. Hereinbelow, the filter device for separating cells is also simply described as “filter device”.

Constitution of the filter container 2 for separating cells is described below.

### <Filter Member-Housing Part>

The filter member-housing part 3 is a tubular member having an opening at both ends. In the filter member-housing part 3, the filter member 11, which will be described later, is housed. Hereinbelow, the filter member-housing part 3 is also simply described as “housing part 3”.

Shape of the housing part 3 is a tubular shape having an opening at both ends. With regard to the tubular shape, the cross-sectional shape in diameter direction may be either a circular shape or a shape other than circular shape such as a polygon. Specific examples of preferred shape of the housing part 3 may include a tubular shape which has a volume of about 0.1 to 400 mL or so, an inner diameter of 0.1 to 15 cm or so, and a thickness of 0.1 to 5 cm or so, and also a rectangular prism shape which is a square type or rectangular type with a single side length of 0.1 to 20 cm or so and a thickness of 0.1 to 5 cm or so.

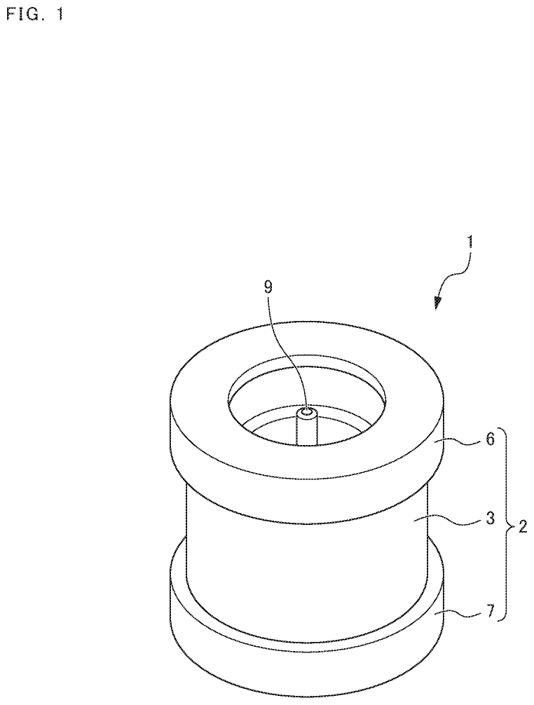

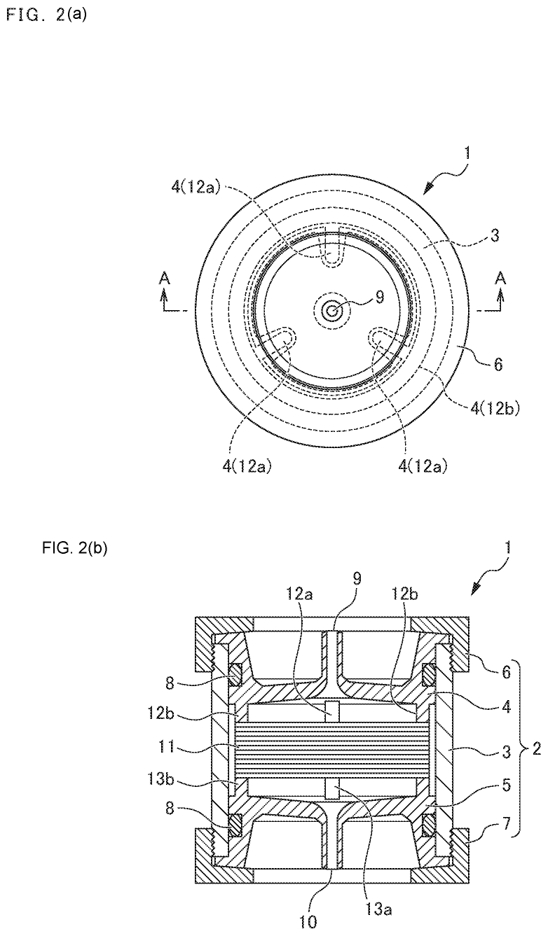

For example, according to one or more embodiments of the container 2 illustrated in FIG. 1, FIG. 2(a), and FIG. 2(b), the tubular container 2 is constituted with the tubular housing part 3, a nozzle-attached inner lid 4 and a nozzle-attached inner lid 5 to put a lid on the openings present at the top and bottom thereof, and an annular outer lid 6 and an annular outer lid 7 to fix the housing part 3, the nozzle-attached inner lid 4, and the nozzle-attached inner lid 5.

The liquid inlet port 9 for introducing a liquid to the inside of the container 2 and the liquid outlet port 10 for discharging a liquid from the container 2 are disposed at the nozzle-attached inner lid 4 and the nozzle-attached inner lid 5, respectively. The liquid inlet port 9 and the liquid outlet port 10 are constituted with a nozzle so as to have easy connection of a tube for transporting a liquid. Shape or size of the nozzle is not particularly limited. Furthermore, although they are referred to as the liquid inlet port 9 and the liquid outlet port 10 for the sake of convenience, in the case of using the filter device 1, it is also possible that a liquid is discharged from the liquid inlet port 9 and a liquid is introduced from the liquid outlet port 10.

The nozzle-attached inner lid 4 and the nozzle-attached inner lid 5 are provided in plug form. Each of the nozzle-attached inner lid 4 and the nozzle-attached inner lid 5 is pushed into the lumen of the housing part 3. In doing so, the nozzle-attached inner lid 4 and the nozzle-attached inner lid 5 are fixed while being in contact with an inner face of the housing part 3. On a contact face between the housing part 3 and each of the nozzle-attached inner lid 4 and the nozzle-attached inner lid 5, a seal 8 may be preferably disposed. By this seal 8, air tightness between the housing part 3 and each of the nozzle-attached inner lid 4 and the nozzle-attached inner lid 5 is ensured so that an attack by microbes or the like from an outside can be prevented. Preferred examples of the seal 8 may include a packing (O ring) made of a resin which is provided around a groove formed on a surface of each of the nozzle-attached inner lid 4 and the nozzle-attached inner lid 5 as illustrated in FIG. 2(b), for example. Arrangement or constitution of the seal 8 is not particularly limited.

It may be possible that each of the nozzle-attached inner lid 4 and the nozzle-attached inner lid 5 is provided so as to be directly fixed to the housing part 3 (not illustrated). By providing a screw, for example, on a surface at which the nozzle-attached inner lid 4 and the nozzle-attached inner lid 5 are in contact with the housing part 3, the nozzle-attached inner lid 4 and the nozzle-attached inner lid 5 can be fixed with the housing part 3. In that case, the annular outer lid 6 and the annular outer lid 7, which are illustrated in FIG. 2(b), become unnecessary.

Inside the container 2, the cell separating material 11 is filled by stacking. For example, by having a filter in which two or more layers of the cell separating material 11 with different fiber diameter are stacked, cell-capturing sites are dispersed, an occurrence of clogging is suppressed, and, simultaneously, separation and collection of cells from the filter can be efficiently carried out. Furthermore, the part at which cell separating materials with the same fiber diameter are continuously stacked is regarded as a single layer without considering the piece number of the stacked cell separating materials.

Furthermore, in the container 2, a washing liquid inlet port (not illustrated) for washing non-adhered cells, which remain independently inside the cell separating material 11, may be provided on the liquid inlet port 9 side. Furthermore, the container 2 may be provided, on the liquid outlet port 10 side, independently with an inlet port for cell-collection liquid (not illustrated) for collecting cells that are captured by the cell separating material 11 (for flowing a cell-collection liquid in an opposite direction to the flow of a cell-containing liquid and a washing liquid).

The container 2 may be produced by using an arbitrary structural material. Specific examples of such structural material may include nonreactive polymers, biocompatible metals, alloys, and glasses. Examples of nonreactive polymers include acrylonitrile polymers such as acrylonitrile butadiene styrene terpolymer (ABS); halogenated polymers such as polytetrafluoroethylene, polychlorotrifluoroethylene, tetrafluoroethylene-hexafluoropropylene copolymer, and polyvinyl chloride; polyamide, polyimide, polysulfone, polycarbonate, polyethylene, polypropylene, polyvinyl chloride-acrylic copolymer, polycarbonate acrylonitrile butadiene styrene, polystyrene, and polymethylpentene. Examples of the metal materials (biocompatible metals and alloys) that are useful as a material of the container include stainless steel, titanium, platinum, tantalum, gold, and alloys thereof, gold plated ferroalloy, platinum plated ferroalloy, cobalt chromium alloy, and titanium nitride-coated stainless steel. Particularly preferred may be the materials having an antimicrobial agent. Specific examples of the materials having an antimicrobial property may include polypropylene, polyvinyl chloride, polyethylene, polyimide, polycarbonate, polysulfone, and polymethylpentene.

### <First Filter-Pressing Part>

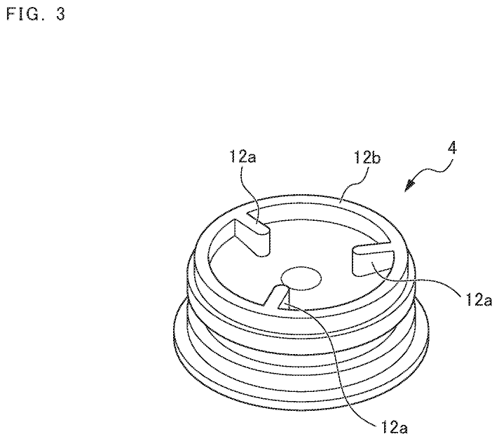

The first filter-pressing part 12 is configured, at a position on the liquid inlet port 9 side of the housing part 3, of three or more first protrusions 12a which protrude from an inner wall face of the housing part 3 or from the vicinity of the inner wall face toward the center or approximate center of a cross-section (cross-section in diameter direction) of an inner space of the housing part 3. Herein, the position of the vicinity of the inner wall face is not particularly limited as long as the effect desired by disposing the first protrusions 12a satisfying pre-determined conditions is obtained. The position of the vicinity of the inner wall face may be, in a cross-section (cross-section in diameter direction) of an inner space of the housing part 3, typically a position at which a distance from the inner wall face is 20% or less of the distance from the center in the cross-section in diameter direction of an inner space of the housing part 3 to the inner wall face. As a specific example in which the first protrusions 12a protrude from the vicinity of the inner wall face of the housing part 3, a case illustrated in FIG. 3, for example, in which the nozzle-attached inner lid 4 is inserted to the housing part 3 and the first filter-pressing part 12 composed of the first protrusions 12a is disposed, can be mentioned. In this case, from the inner thickness side position of a first annular support 12b provided in the nozzle-attached inner lid 4 rather than the inner wall face of the housing part 3, the first protrusions 12a protrude toward the center or approximate center of a cross-section (cross-section in diameter direction) of an inner space of the housing part 3. In addition, in FIG. 2(a) and FIG. 3, the illustration has been made for a case in which three first protrusions 12a exist.

The center of a cross-section (cross-section in diameter direction) of an inner space of the housing part 3 means a center position of a cross-sectional shape. The center of a cross-section (cross-section in diameter direction) of an inner space of the housing part 3 is a center of a circle if the cross-sectional shape is circular, or it is an intersection point of diagonals if the cross-sectional shape is square. The approximate center of a cross-section (cross-section in diameter direction) of an inner space of the housing part 3 is not particularly limited as long as it is an area near the center. The range of the approximate center may be, so to speak, a circular area with the aforementioned center at the center, and it indicates a range of a circle which has an area of 50% by area or so relative to the area of a cross-section (cross-section in diameter direction) of an inner space of the housing part 3.

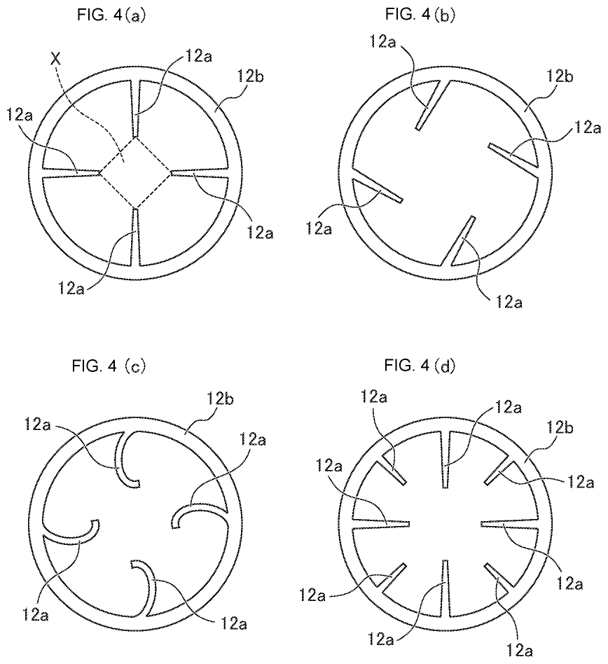

Specific examples of one or more embodiments of the protrusion of the first protrusion 12a are illustrated in FIG. 4(a) to FIG. 4(d). In FIG. 4(a), an embodiment in which plural first protrusions 12a with the same length protrude straightforward toward the center of a cross-section (cross-section in diameter direction) of an inner space of the housing part 3 is illustrated. In FIG. 4(b), an embodiment in which plural first protrusions 12a with the same length protrude straightforward toward the approximate center area of a cross-section (cross-section in diameter direction) of an inner space of the housing part 3, but toward a direction offset from the center, is illustrated. Furthermore, as illustrated in FIG. 4(c), the first protrusions 12a may also protrude while being curved. In that case, the shape of the first protrusion 12a observed from a direction perpendicular to the cross-section in diameter direction of the housing part 3 may be an arc shape, an S shape, or a zigzag shape, for example. Furthermore, as illustrated in FIG. 4(d), plural first protrusions 12a may include plural protrusions having different lengths. Among them, as illustrated in FIG. 4(a) and FIG. 4(b), it may be preferable that plural first protrusions 12a protrude straightforward toward the center of a cross-section (cross-section in diameter direction) of an inner space of the housing part 3, and it may be more preferable that plural first protrusions 12a with the same length protrude straightforward toward the center of a cross-section (cross-section in diameter direction) of an inner space of the housing part 3 as illustrated in FIG. 4(a).

The number of the first protrusions 12a is not particularly limited, as long as it is 3 or higher. From the viewpoint of easily increasing the desired cell collection rate, the number of the first protrusions 12a may be preferably 5 or higher and 12 or lower, and more preferably 6 or higher and 10 or lower. In the first filter-pressing part 12, plural first protrusions 12a may be arranged at a regular interval or an irregular interval. From the viewpoint of easy and even permeation of a liquid containing cells to the filter member 11, it may be preferable that plural first protrusions 12a are arranged at a regular interval in the first filter-pressing part 12.

The first filter-pressing part 12 is disposed such that, when the filter member 11 to be described later is housed in the housing part 3, the first filter-pressing part 12 is in contact with the filter member 11 while deforming the filter member 11.

Shape of the cross-section of the first protrusion 12a constituting the first filter-pressing part 12, in which the cross-section means a cross-section perpendicular to the protrusion direction of the first protrusion 12a, is not particularly limited. Typically, from the viewpoint of having easy contact between the first protrusion and the filter member 11 in large area, it may be preferably a rectangular shape in which one side is in contact with the filter member 11.

When the filter member 11 is housed in the housing part 3, the filter member 11 is brought into contact with the first filter-pressing part 12 while being supported by the second filter-pressing part 13 to be described later. As a result, the surface in contact with the first protrusion 12a of the filter member 11 is pushed in with depression by the first protrusion 12a constituting the first filter-pressing part 12. As a result, near the area that is in contact with the first protrusion 12a of the filter member 11, the filter member 11 is compressed. On the other hand, in the area not in contact with the first protrusion 12a of the filter member 11, the filter member 11 becomes loose. This loosening phenomenon is caused by the elasticity (repulsive force) of the filter member 11, and the loosening degree partially varies depending on a distance from the contact area in the surface layer. Accordingly, in an area corresponding to a polygon formed by the tips of plural first protrusions 12a in the surface layer of the filter member 11, the density becomes low near the surface layer of the filter member 11. On the other hand, in the area in contact with plural first protrusions 12a or the area adjacent thereto in the surface layer of the filter member 11, high density is yielded near the surface layer of the filter member 11.

It is believed that, as a sparse and dense pattern is formed in the surface layer of the filter member 11, control of collection rate depending on a size of cells as a separation subject can be easily carried out. Specifically, in an area in which the filter member 11 is sparse, wide pores may be yielded near the surface layer in the filter member 11 while the pore size does not change near the center part in the thickness direction of the filter member 11. Accordingly, cells with small size can be drawn near to the center part in the thickness direction of the filter member 11. On the other hand, near the surface layer of the filter member 11, cells with large size can be easily captured in pores with large size.

As a result, when a collection liquid is introduced to the filter device 1 for separating cells after capturing cells, large cells near the surface layer of the filter member 11 can be easily released into the collection liquid while small cells near the center part in the thickness direction of the filter member 11 are not likely to get released into the collection liquid.

For the reasons described above, it is presumed that the collection rate of cells with desired size can be increased by having the area of a polygon formed by the tips of plural first protrusions 12a set at pre-determined ratio, which will be described later, relative to the area of a cross-section in diameter direction of an inner space of the housing part 3.

Specifically, when the area of a polygon formed by connecting each tip of the three or more first protrusions 12a constituting the first filter-pressing part 12 is A1 and the area of a cross-section (cross-section in diameter direction) of an inner space of the housing part 3 at the position of the first protrusion 12a is A2, the first protrusion 12a is provided such that the area ratio R1 that is calculated by the following equation is 6 to 50%:

Area ratio R1(%)=A1/A2×100.

The area ratio R1 may be more preferably 6 to 40%, even more preferably 6 to 30%, and particularly preferably 7 to 28%.

As described in the above, as the filter member 11 is pushed in with depression by the first protrusions 12a, a sparse and dense pattern is formed in the surface layer of the filter member 11. Furthermore, as the area ratio R1 is 6 to 50%, not only the cells with large size can be easily captured by pores with large size near the surface layer of the filter member 11 but also easiness of holding the cells with small size near the center part in thickness direction of the filter member 11 can be controlled at a suitable level. In addition, the sparse and dense pattern is strongly related with securement of a liquid flow path. Specifically, if R1 is less than 6%, a dense region is excessively wide in the surface layer of the filter member 11 so that the liquid flow path is limited only to a partial sparse region. As a result, a drift flow is caused and the filter effect is not obtained. On the other hand, if R1 is more than 50%, contrast between the sparsity and density is small in the surface layer of the filter member 11, and the loose region becomes excessively wide. Due to this reason, cells with large size pass through the wide pores that are present near the surface layer of the filter member 11. As a result, regardless of the degree of size, the cells are kept within a much inner part in the thickness direction so that the cells cannot be collected or control of the collection rate depending on a size of the cells as a separation subject is difficult to achieve.

The first filter-pressing part 12 may be disposed such that it directly protrudes from an inner wall of the housing part 3. From the viewpoint of easy housing of the filter member 11 in the container 2, the first filter-pressing part 12 may be preferably disposed such that it is constituted with the first annular support 12b and the first protrusion 12a on a tip surface of the nozzle-attached inner lid 4 to be inserted to an inner space of the housing part 3 of the container 2, as it is illustrated in FIG. 3. The first protrusion 12a is supported by the first annular support 12b as illustrated in FIG. 3.

As described in the above, in a case in which the first filter-pressing part 12 is disposed on the nozzle-attached inner lid 4, square X represented by dotted line in FIG. 4(a) corresponds to a polygon formed by connecting each tip of plural first protrusions 12a. The area of the square X corresponds to the aforementioned A1. Meanwhile, the area of a circle corresponding to the outer circumference of the first annular support 12b in FIG. 4(a) is equivalent to A2, which is the area of a cross-section (cross-section in diameter direction) of an inner space of the housing part 3 at the position of the first protrusions 12a.

### <Second Filter-Pressing Part>

Shape of the second filter-pressing part 13 is not particularly limited as long as the filter member 11 can be supported in a state in which liquid transport from a single surface of the filter member 11 to the other surface is allowed when the filter member 11 is housed in the container 2. Specific examples of the second filter-pressing part 13 satisfying the above conditions may include a mesh, a perforated plate for drain, or the like.

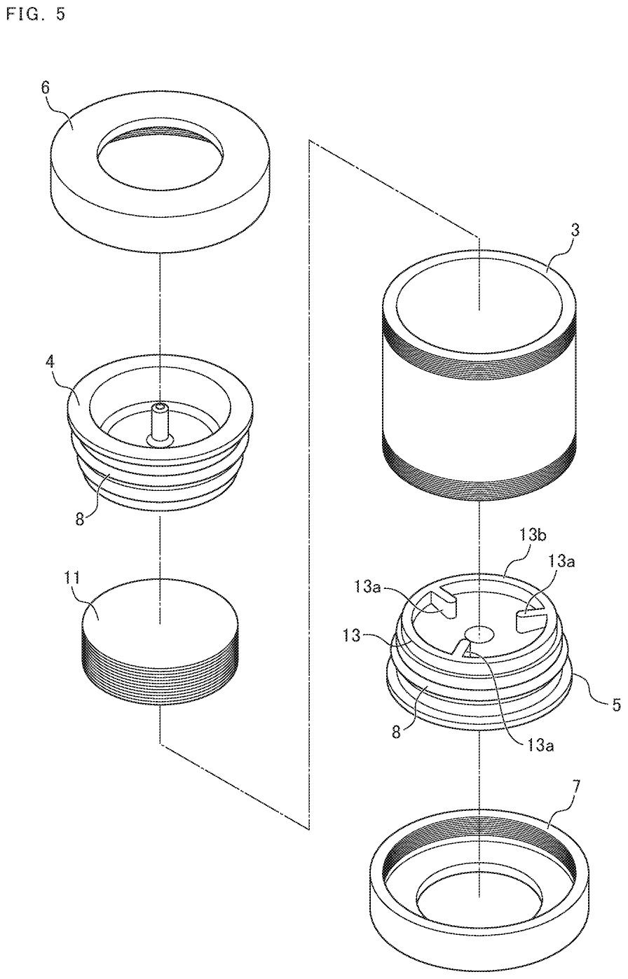

When a liquid containing cells is flown through the filter device 1, from the viewpoint of having easy and good flow of the liquid from the liquid inlet port 9 to the liquid outlet port 10, or from the liquid outlet port 10 to the liquid inlet port 9, the second filter-pressing part 13 may be preferably configured, at a position on the liquid outlet port 10 side of the housing part 3, of three or more second protrusions 13a which protrude from the inner wall face of the housing part 3 or the vicinity of the inner wall face toward the center or approximate center of a cross-section (cross-section in diameter direction) of an inner space of the housing part 3, similar to the first filter-pressing part 12. A typical example of the vicinity of the inner wall face of the housing part 3 may be the same as those described for the first filter-pressing part 12. In FIG. 5, the filter device 1 or the container 2, which is provided with the second filter-pressing part 13 configured of three or more second protrusions 13a, is illustrated.

In a case in which the second filter-pressing part is configured of three or more second protrusions 13a, the method or shape of the second protrusion 13a may be preferably the same as the method or shape described for the first protrusion 12a. Furthermore, when the area of a polygon formed by connecting each tip of the three or more second protrusions 13a constituting the second filter-pressing part 13 is A3 and the area of a cross-section (cross-section in diameter direction) of an inner space of the housing part 3 at the position of the second protrusions 13a is A4, the second protrusions 13a may be preferably provided such that the area ratio R2 that is calculated by the following equation is 6 to 50%:

Area ratio R2(%)=A3/A4×100.

The area ratio R2 may be more preferably 6 to 40%, even more preferably 6 to 30%, and particularly preferably 7 to 28%. If the R2 is lower than 6% or higher than 50%, there may be a case in which the same problems as those described before for a case in which R1 is lower than 6% or higher than 50% may occur depending on the value of the aforementioned R1.

By constituting the filter device 1 for separating cells according to housing of the filter member 11 to be described later in the filter container 2 for separating cells described in the above, desired cells can be easily collected at a high collection rate.

### <<Filter Device for Separating Cells>>

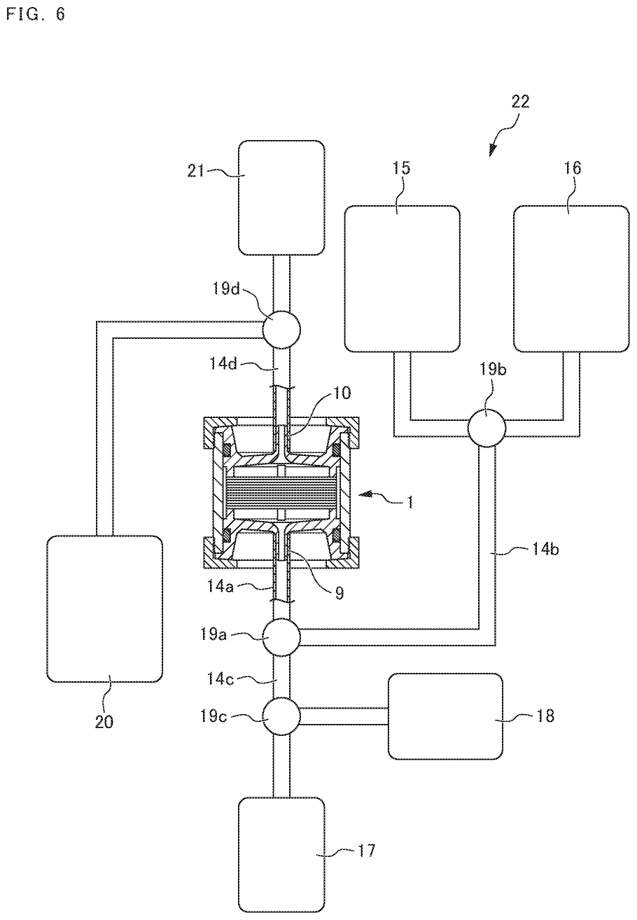

The filter device 1 for separating cells is constituted by housing the filter member 11 in the aforementioned filter container 2 for separating cells as described in the above. In FIG. 6, a brief exploded view of the filter device 1 for separating cells is illustrated for a case in which the aforementioned filter container 2 for separating cells is constituted with the housing part 3, the nozzle-attached inner lid 4, the nozzle-attached inner lid 5, the annular outer lid 6, and the annular outer lid 7. Hereinbelow, descriptions are given for the filter member 11.

### <Filter Member>

Form of the filter member 11 is not particularly limited and examples thereof include a porous body having communication pore structure, a fiber aggregate, and a fabric. Preferably, it may be a woven fabric or a non-woven fabric composed of fibers, and it may be more preferably a non-woven fabric.

As a material of the filter member 11, polyolefin (for example, polypropylene, polyethylene, high density polyethylene and low density polyethylene, or the like), polyester, vinyl chloride, polyvinyl alcohol, vinylidene chloride, rayon, vinylon, polystyrene, an acrylic resin (for example, polymethyl methacrylate, polyhydroxyethyl methacrylate, polyacrylonitrile, polyacrylic acid, polyacrylate, or the like), nylon (for example, aliphatic polyamide and aromatic polyamide (aramid)), polyurethane, polyimide, cupra, kevlar, carbon, phenolic resin, tetoron, pulp, linen, cellulose, kenaf, chitin, chitosan, glass, cotton, or the like can be mentioned. Among them, a polymer such as polyester, polypropylene, acryl, rayon, nylon, polybutylene terephthalate, polyethylene terephthalate or the like can be appropriately used. Among those materials, the filter member 11 may be composed of a single material or a composite material in which plural materials are combined.

The average particle diameter of the filter member 11 can be suitably selected depending on a type of the desired cell, and it is not particularly limited.

In order to further improve the performance of the filter member 11, the filter member 11 may be subjected to a hydrophilic treatment. According to the hydrophilic treatment, effects like suppression of non-specific capturing of cells other than desired necessary cells, enhancement of the performance of passing the cell-containing liquid through the filter member 11 without any bias, enhancement of the collection rate of necessary cells, or the like can be provided.

Examples of the hydrophilic treatment method include: a method of adsorbing a water-soluble polyhydric alcohol, a compound having a hydroxyl group, a cationic group, or an anionic group, or a copolymer thereof (for example, a copolymer of monomer including hydroxyethyl methacrylate, dimethylaminoethyl methacrylate, or the like);

a method of adsorbing a water-soluble polymer (polyethylene glycol, polyvinyl pyrrolidone, polyvinyl alcohol or the like);
a method of immobilizing a hydrophilic polymer to a hydrophobic membrane (for example, a method of chemically binding a hydrophilic monomer to a surface or the like);
a method of applying electron beams;
a method of cross-linking and insolubilizing a hydrophilic polymer by applying radiation beams to a cell separation filter in water-containing state;
a method of insolubilizing and immobilizing a hydrophilic polymer by heat treatment in dry state;
a method of sulfonizing a surface of a hydrophobic membrane;
a method of forming a membrane from a mixture of a hydrophilic polymer and a hydrophobic polymer dope;
a method of imparting a hydrophilic group to a membrane surface by treatment with an aqueous alkali (such as NaOH and KOH) solution;
an insolubilization method in which a hydrophobic porous membrane is immersed in an alcohol, then is treated with an aqueous water-soluble polymer solution, and is dried, followed by heat treatment, treatment with radiation beams or the like; or
a method of adsorbing a substance having surfactant activity.

Examples of the hydrophilic polymer include polyvinyl pyrrolidone, polyvinyl alcohol, polyethylene glycol, ethylene-vinyl alcohol copolymer, polyhydroxyethyl methacrylate, polysaccharides (cellulose, chitin, chitosan, or the like), and water-soluble polyhydric alcohol.

Examples of the hydrophobic polymer include polystyrene, polyvinyl chloride, polyolefin (polyethylene, polypropylene, or the like), acryl, urethane, vinylon, nylon, and polyester.

In order to enhance the adhesion property of the cells desired for collection to a filter member, it is also possible to immobilize on top of a filter member a cell adhesive protein or an antibody for a specific antigen expressed in desired stem cells. Examples of the cell adhesive protein include fibronectin, laminin, vitronectin, and collagen. Examples of the antibody include CD73, CD90, CD105, CD166, CD140a, and CD271, but it is not limited thereto. For example, as a method for immobilization, a method like a cyanogen bromide activation method, an azide acid derivative method, a condensing reagent method, a diazo method, an alkylation method, and a cross-linking method, which are a general method for protein immobilization, can be arbitrarily used.

The thickness of the filter member 11 is not particularly limited. The thickness of the filter member 11 is suitably determined in consideration of the aforementioned gap between the first filter-pressing part 12 and the second filter-pressing part 13. Typically, the thickness of the filter member 11 may be preferably 3 to 20 mm, and more preferably 5 to 10 mm. The open diameter of the filter member 11 can be defined by a product (air permeability coefficient M) of the air permeability (cc/cm² sec) and thickness (mm). The range may be preferably 7.0 or more and 14.2 or less, and most preferably 9.2 or more and 10.0 or less.

### <<Method for Obtaining Cell-Containing Liquid Containing Desired Cells>>

According to a treatment using the aforementioned filter device for separating cells, a cell-containing liquid containing desired cells can be obtained.

### <Cells>

Cells as a subject for obtainment are not particularly limited. Examples thereof include pluripotent biological stem cells such as induced pluripotent stem cells (iPS cells), embryonic stem cells (ES cells), mesenchymal stem cells, adipose-derived mesenchymal cells, adipose-derived stromal stem cells, pluripotent adult stem cells, bone marrow stroma cells, and hematopoietic stem cells; lymphoid cells such as T cells, B cells, killer T cells (cytotoxic T cells), NK cells, NKT cells, and regulatory T cells; macrophages, monocytes, dendritic cells, granulocytes, red blood cells, platelets, somatic cells such as nerve cells, muscle cells, fibroblasts, liver cells, and myocardial cells; and cells subjected to treatments such as gene incorporation or differentiation.

Preferred among those cells may be white blood cells, hematopoietic stem cells, and/or mononuclear cells from the viewpoint of significant exhibition of the effect of enhancing the collection rate, and monocytes may be more preferable. Furthermore, examples of the white blood cells include granulocytes in peripheral blood such as neutrophils, eosinophils, or basophils, and mononuclear cell such as monocytes and lymphocytes. Hereinbelow, the method for obtaining a cell-containing liquid containing monocytes is described as a representative example.

### <Method for Obtaining Cell-Containing Liquid Containing Monocytes>

A preferred method for obtaining a cell-containing liquid containing monocytes may be a method including:

capturing monocytes by the filter member 11 by supplying, from the liquid inlet port 9, a crude cell-containing liquid containing monocytes and small cells, which are cells having smaller average size than monocytes, to the inside of the aforementioned filter device 1 for separating cells followed by passing the crude cell-containing liquid through the filter device 1 for separating cells;
yielding the cell-containing liquid by releasing, in a collection liquid, the monocytes captured by the filter member 11 by supplying a collection liquid to the inside of the filter device 1 for separating cells; and
collecting the cell-containing liquid from the inside of the filter device 1 for separating cells.

As for the crude cell-containing liquid, use can be made without any particular limitation as long as it is a suspension containing at least monocytes and cells including small cells as a cell having smaller average size than monocyte. Specific examples of the suitable small cells include granulocytes (neutrophils, eosinophils, and basophils), lymphocytes, red blood cells, and platelets. For example, a suspension after carrying out an enzyme treatment, a disruption treatment, an extraction treatment, a decomposition treatment, an ultrasonication treatment or the like of a living tissues like umbilical cord, blood or body fluid like bone marrow fluid and umbilical cord blood, a cell suspension obtained by a pre-treatment such as density gradient centrifugal treatment, filtration treatment, enzyme treatment, decomposition treatment, and ultrasonication treatment of blood or bone marrow fluid, or the like are exemplified as a crude cell-containing liquid. Furthermore, the crude cell-containing liquid may be a suspension obtained after performing culture, proliferation, or the like of cells such as the white blood cells by using a culture liquid, a stimulation factor, or the like. Herein, the expression “crude cell-containing liquid” means a cell-containing liquid that is provided to a treatment for obtaining cells by using the filter device 1 for separating cells.

When a crude cell-containing liquid is introduced from the liquid inlet port 9 of the filter device 1 for separating cells, it may be favorable to generate a difference in pressure between the liquid inlet port 9 and the liquid outlet port 10. The method for generating a difference in pressure is not particularly limited. Examples of the method include a method of introducing a crude cell-containing liquid to the filter device 1 for separating cells either by applying pressure to a crude cell-containing liquid or by utilizing a difference in gravity, a method of suctioning a crude cell-containing liquid to the filter device 1 for separating cells after depressurizing the liquid outlet port 10 side, or the like. Accordingly, a crude cell-containing liquid is supplied to the filter device 1 for separating cells, and by contacting the crude cell-containing liquid with the filter member 11, monocytes are captured by the filter member 11. Degree of the difference in pressure is not particularly limited as long as it does not allow an occurrence of a breakage of the container 2 or filter member 11 or an occurrence of an excessive breakage of cells that are contained in a crude cell-containing liquid. As for the filter member 11, it may be preferable to use those capable of capturing monocytes.

Subsequently, by supplying a collection liquid to the filter member 11 in which monocytes are captured, the monocytes are released from the filter member 11 to a collection liquid so that a cell-containing liquid containing monocytes is generated. By introducing this collection liquid containing white blood cells or the like from the liquid inlet port 9 to a bag or the like which is exclusively used for collection, white blood cells or the like can be collected. The collection liquid is introduced from the liquid outlet port 10 or the liquid inlet port 9 to the filter device 1 for separating cells. Furthermore, the method of collecting the cell-containing liquid containing monocytes from the inside of the filter device 1 for separating cells is not particularly limited. Typically, a cell-containing liquid containing monocytes may be collected from the liquid inlet port 9. From the viewpoint that the monocytes captured by the filter member 11 can be easily released into a collection liquid in accordance with a flow of a collection liquid, it may be preferable that the collection liquid is introduced to the filter device 1 for separating cells such that it passes through the filter member 11 from the liquid outlet port 10. In this case, it may be preferable to carry out so-called reverse-washing operation. The reverse-washing operation is an operation in which, while introducing a collection liquid from the liquid outlet port 10, the cell-containing liquid containing monocytes is directly collected from the liquid inlet port 9.

The collection liquid is not particularly limited as long as it is isotonic to cells. Specific examples of the collection liquid may include a liquid which has a record of being used as an injection solution like physiological saline and ringer solution, a buffer solution, and a medium for cell culture. In particular, when use of a cell-containing liquid involves a culture step, a medium allowing direct culture may be preferable. When a cell-containing liquid can be directly used for a treatment without involving any culture step, a collection liquid with guaranteed safety such as an isotonic solution, which has a record of being used for dropwise addition like physiological saline, may be preferably used.

The operation described in the above may be carried out under room temperature, or it may be carried out under refrigerating temperature. Examples of the operation which is carried out under refrigerating temperature include a treatment of refrigerated crude cell-containing liquid. As for the storage of a crude cell-containing liquid, storage in a refrigerator set at refrigerating temperature, storage in water bath, storage using dry ice, or the like can be mentioned. From the viewpoint of universality, storage using a refrigerator may be preferable. The refrigerating temperature may be preferably 1° C. or higher and 6° C. or lower, and more preferably 3° C. or higher and 5° C. or lower. There is a possibility that, when the refrigerating temperature is lower than 1° C., cell death is yielded, and, when the refrigerating temperature is higher than 6° C., contamination occurs as caused by bacterial growth.

Furthermore, from the viewpoint of achieving easy capture of monocytes by having a neat state of the filter member 11, it is also possible that, before supplying a crude cell-containing liquid to the filter device 1 for separating cells, physiological saline or a buffer solution is introduced from the liquid inlet port 9 or the liquid outlet port 10 to the filter device 1 for separating cells, and then the filter member 11 is brought into contact with physiological saline or a buffer solution.

Before generating a cell-containing liquid containing monocytes by using a collection liquid, it is possible to remove impurity components in the filter by having physiological saline or a buffer solution introduced from the liquid inlet port 9 and discharged from the liquid outlet port 10. Accordingly, it becomes possible to reduce the impurity components in cells (monocytes) to be collected.

### <<Method for Producing Dendritic Cell-Containing Liquid Used for Dendritic Cell Therapy>>

By using the aforementioned filter device 1 for separating cells, a dendritic cell-containing liquid used for dendritic cell therapy can be produced. According to the methods, a cell-containing liquid containing monocytes is obtained first by the aforementioned method for obtaining a cell-containing liquid containing monocytes. Subsequently, the monocytes contained in the obtained cell-containing liquid are differentiated into dendritic cells in accordance with various known methods. Typically, a protein kit or a medium for inducting differentiation into dendritic cells may be commercially available, and dendritic cells may be induced from monocytes by using them.

### EXAMPLES

Hereinbelow, one or more embodiments of the present invention are described in detail using Examples, but the present invention is not limited to the following Examples.

### Examples 1 to 6, Comparative Example 1, and Comparative Example 2

As illustrated in FIG. 5, in the tubular housing part 3 having a height (inner side size of 5 mm or 12 mm) and a diameter (inner diameter of 60 mm or 45 mm) shown in Table 1, 50 pieces or 105 pieces of a polyester non-woven fabric (weight per unit area: 35 g/m², filter member 11), which have been cut to a round shape with diameter of 60 mm or 45 mm, were filled. Subsequently, to the openings at the top and bottom of the housing part 3, the nozzle-attached inner lid 4 provided with a first filter-pressing part and the nozzle-attached inner lid 5 provided with a second filter-pressing part were inserted. The nozzle-attached inner lid 4 and the nozzle-attached inner lid 5 were screw-fixed from the above by the annular outer lid 6 and the annular outer lid 7, and thus the filter device 1 for separating cells having a structure illustrated in FIGS. 1, 2(a), and 2(b) was produced.

Furthermore, on the nozzle-attached inner lid 4 and the nozzle-attached inner lid 5, a first filter-pressing part and a second filter-pressing part, both having the same shape, were disposed. In addition, the first protrusion 12a and the second protrusion 13a are provided such that they protrude straightforward from the first annular support 12b and the second annular support 13b, respectively, toward the center of a cross-section in diameter direction of the housing part 3. Furthermore, in the first filter-pressing part and the second filter-pressing part, plural first protrusions 12a and plural second protrusions 13a are provided such that each of them are present at the regular interval.

Shape of the first filter-pressing part 12 and the second filter-pressing part 13 illustrated in FIG. 2(a), FIG. 3, and FIG. 5 is just one example, and number, shape, and length of the first protrusion 12a and the second protrusion 13a that are contained in each of the first filter-pressing part 12 and the second filter-pressing part 13 used in Examples are not limited to the shape illustrated in FIG. 2(a), FIG. 3, and FIG. 5.

Next, by connecting the circuit illustrated in FIG. 6 to the liquid inlet port 9 and the liquid outlet port 10 of the filter device 1 for separating cells, a device 22 for separating cells was produced.

In the circuit illustrated in FIG. 6, tube 14a is connected to the liquid inlet port 9. To the tube 14a, tube 14b connected to a means 15 for holding a cell suspension and a means 16 for holding physiological saline for priming, and tube 14c connected to a means (collection bag) 17 for holding a collection liquid after passing through the filter and a means 18 for collecting a collection liquid collected in a collection bag or the like are connected via a flow path switching means 19c. To the tube 14b, the means 15 for holding a cell suspension and the means 16 for holding physiological saline used for priming or column washing are connected via a flow path switching means 19b. To the tube 14c, the means 17 for holding a collection liquid after passing through the filter and the means 18 for collecting a collection liquid collected in a collection bag or the like are connected via the flow path switching means 19c path. Furthermore, tube 14d is connected to the liquid outlet port 10, to which a means (waste liquid bag) 20 for holding a cell suspension after passing through the filter and a means 21 for collecting a collection liquid are connected via a flow path switching means 19d.

By using the device 22 for separating cells, the operation of separating cells (monocytes) was carried out. Furthermore, the operation of each flow path switching means was suitably carried out depending on a type of a liquid to be flown through the filter device 1 for separating cells and a desired means for liquid transport. First, by using 50 mL to 150 mL of physiological saline of the means 16 for holding physiological saline, the priming operation of the filter device 1 for separating cells was carried out. After that, in the waste liquid bag 20, physiological saline after passing through the filter device 1 for separating cells was collected. Next, from the means 15 for holding a cell suspension, 100 mL of a solution with concentrated white blood cells (pig blood prepared by anti-coagulation using CPD) was allowed to flow through the filter device 1 for separating cells by using gravitational force. After that, the pass-through solution was collected in the waste liquid bag 20. The solution with concentrated white blood cells which has been used for the above operation was produced from buffy coat resulting from centrifuge (3000 rpm, for 30 minutes) of pig blood prepared by anti-coagulation using CPD, in which the production was made such that the number of white blood cells to be treated is 1.0×10⁹ cells to 4.0×10⁹ cells. After that, by using the flow path switching means 19b, 100 mL of physiological saline of the means 16 for holding physiological saline was allowed to flow through the filter device 1 for separating cells by using gravitational force. Subsequently, the pass-through solution was collected in the waste liquid bag 20. Finally, 50 mL of physiological saline was introduced, at a flow rate of 10 to 50 mL/second from the liquid outlet port 10 of the filter device 1 for separating cells, manually by using a syringe as a means 21 for collecting the collection liquid. Subsequently, the collection liquid (cell-containing liquid) was collected in a collection bag 17 connected to the liquid inlet port 9. Concentration of the white blood cells in the collection liquid (cell-containing liquid) and cell suspension (crude cell-containing liquid) before the treatment was measured by a hemocyte counter (K-4500, SYSMEX CORPORATION). Furthermore, with regard to the monocyte concentration, fluorescent labeling was carried out for CD14 as a cell surface marker, and the measurement was carried out by the hemocyte counter and a flow cytometer (FACS canto, BD Biosciences). After that, the number of the white blood cells and the number of the monocytes were calculated from the volume of the cell suspension before the treatment and also the collection liquid, and the white blood cell collection rate and monocyte collection rate were obtained, respectively. The results are shown in Table 1.

TABLE 1

| | | Example | | | | | | Comparative Example | |
|---|---|---|---|---|---|---|---|---|---|
| | | 1 | 2 | 3 | 4 | 5 | 6 | 1 | 2 |
| Shape of filter | Height (mm) | 5 | 5 | 5 | 5 | 5 | 5 | 12 | 5 |
| member-housing part | Inner diameter (mm) | 60 | 60 | 60 | 60 | 60 | 60 | 45 | 60 |
| | Cross-sectional area | 2826 | 2826 | 2826 | 2826 | 2826 | 2826 | 1590 | 2826 |
| | of inner space A2 (mm²) | | | | | | | | |
| Shape of first | Number of protrusions | 9 | 10 | 10 | 9 | 9 | 9 | 3 | 9 |
| pressing part/second | Length of protrusion (mm) | 15 | 11 | 15 and 11 | 19 | 11 and 7 | 19 and 6 | 12 | 22 |
| pressing part | Area of polygon A1 (mm²) | 472 | 766 | 656 | 221 | 1029 | 865 | 83 | 85 |

| | | | | | | | | |
|---|---|---|---|---|---|---|---|---|
| A1/A2 (Area %) | 17 | 27 | 23 | 7 | 36 | 31 | 5 | 3 |
| Monocyte collection rate (%) | 75 | 50 | 49 | 55 | 52 | 83 | 29 | 29 |
| White blood cell collection rate (%) | 62 | 51 | 38 | 52 | 47 | 66 | 66 | 49 |

According to Table 1, when the filter device 1 for separating cells of Examples which is constituted such that a pre-determined area ratio R1 (A1/A2 (% by area)) is 6 to 50%, the monocytes can be collected at a high collection rate (in addition, monocytes can be collected at high purity). On the other hand, when the filter device 1 for separating cells which is constituted such that a pre-determined area ratio R1 (A1/A2 (% by area)) is not within 6 to 50%, it was recognized that the monocytes are collected only at a low collection rate and low purity.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

### EXPLANATION OF REFERENCE NUMERALS

1 FILTER DEVICE FOR SEPARATING CELLS
2 FILTER CONTAINER FOR SEPARATING CELLS
3 FILTER MEMBER-HOUSING PART
4, 5 NOZZLE-ATTACHED INNER LID
6, 7 ANNULAR OUTER LID
8 SEAL
9 LIQUID INLET PORT
10 LIQUID OUTLET PORT
11 FILTER MEMBER
12 FIRST FILTER-PRESSING PART
12a FIRST PROTRUSION
12b FIRST ANNULAR SUPPORT
13 SECOND FILTER-PRESSING PART
13a SECOND PROTRUSION
13b SECOND ANNULAR SUPPORT

What is claimed is:

1. A filter container for separating cells, comprising:
a liquid inlet port;
a liquid outlet port;
a filter member-housing part;
a first filter-pressing part; and
a second filter-pressing part,
wherein the filter member-housing part is a tubular member having an opening at each longitudinal end,
wherein the liquid inlet port is provided on a first opening end side of the filter member-housing part, and the liquid outlet port is provided on a second opening end side of the filter member-housing part,
wherein the first filter-pressing part is located on the first opening end side and comprises three or more first protrusions protruding from an inner wall face of the filter member-housing part, or protruding from the vicinity of the inner wall face, toward a center or approximate center of a cross-section of an inner space of the filter member-housing part,
wherein the first filter-pressing part is disposed such that, when a filter member is housed in the filter member-housing part, the first filter-pressing part is in contact with the filter member while deforming the filter member,
wherein the second filter-pressing part is located on the second opening end side such that the filter member is interposed between the first filter-pressing part and the second filter-pressing part, and
wherein an area ratio R1 that is calculated by the following equation is 6 to 50%:
Area ratio R1(%)=A1/A2×100%,
where A1 is an area of a polygon formed by connecting each tip of the three or more first protrusions, and A2 is a cross-sectional area of the inner space of the filter member-housing part where the three or more first protrusions are located.

2. The filter container according to claim 1, wherein the second filter-pressing part comprises three or more second protrusions protruding from the inner wall face of the filter member-housing part, or protruding from the vicinity of the inner wall face, toward a center or approximate center of a cross-section of the inner space of the filter member-housing part.

3. The filter container according to claim 2, wherein an area ratio R2 that is calculated by the following equation is 6 to 50%:
Area ratio R2(%)=A3/A4×100%,
where A3 is an area of a polygon formed by connecting tips of the three or more second protrusions of the second filter-pressing part, and A4 is a cross-sectional area of the inner space of the filter member-housing part where the three or more second protrusions are located.

4. The filter container according to claim 1, wherein the area ratio R1 is 6 to 40%.

5. The filter container according to claim 3, wherein the area ratio R2 is 6 to 40%.

6. The filter container according to claim 1, wherein the number of the first protrusions is 5 to 12.

7. The filter container according to claim 3, wherein the number of the second protrusions is 5 to 12.

8. A filter device for separating cells, comprising the filter container for separating cells according to claim 1 and the filter member, wherein
the filter member is housed in the filter member-housing part.

9. The filter device according to claim 8, wherein the filter member consists of a non-woven fabric.

10. A method for obtaining a cell-containing liquid containing monocytes, comprising:
supplying, from the liquid inlet port of the filter container, a crude cell-containing liquid containing monocytes and small cells having a smaller average size than monocytes to an inside of the filter device according to claim 8;
passing the crude cell-containing liquid through the filter member of the filter device to capture the monocytes on the filter member;
supplying a collection liquid to the inside of the filter device to release the captured monocytes into the collection liquid; and
collecting the collection liquid from the inside of the filter device, wherein the collection liquid comprises the released monocytes.

11. The method according to claim 10, wherein the collection liquid is supplied from the liquid outlet port and collected from the liquid inlet port.

12. A method for producing a dendritic cell-containing liquid used for dendritic cell therapy, comprising:
supplying, from the liquid inlet port to the filter container, a crude cell-containing liquid containing monocytes and small cells having a smaller average size than monocytes to an inside of the filter device according to claim 8;
passing the crude cell-containing liquid through the filter member of the filter device to capture the monocytes on the filter member;
supplying a collection liquid to the inside of the filter device to release the captured monocytes into the collection liquid;
collecting the collection liquid from the inside of the filter device, wherein the collection liquid comprises the released monocytes; and
differentiating the monocytes contained in the collection liquid into dendritic cells.

13. The method according to claim 12, wherein the collection liquid is supplied from the liquid outlet port and collected from the liquid inlet port.
